(19) United States
(12) Patent Application Publication
(10) Pub. No.: US 2020/0246580 A1
(43) Pub. Date: Aug. 6, 2020

(54) Systems, Methods, and Articles For Behavioral Disorder Monitoring and Treatment

(71) Applicant: Alexander Asher Dott, (US)

(72) Inventor: Alexander Asher Dott, Stuart, FL (US)

(73) Assignee: Alexander Asher Dott, Stuart, FL (US)

(21) Appl. No.: 16/788,803

(22) Filed: Feb. 12, 2020

Related U.S. Application Data
(60) Provisional application No. 62/800,552, filed on Feb. 3, 2019.

(51) Int. Cl. A61M 21/02

(52) U.S. Cl. CPC A61M 21/02; A61M 2021/0016

(57) ABSTRACT

Systems, methods, and articles are disclosed for the behavioral treatment of an individual. One embodiment comprising a wearable housing assembly further comprising a microcontroller, charging jack port, status monitor, selection element, sensor array, and deterring member attached to the user via a connection strap, adjustment belt, frame, prong, and adjustment hole. The user selects the input/output properties of the microcontroller, status monitor, sensor array, and deterring member using the selection element. Upon performing an undesirable emotional outburst, the user is automatically administered a deterrence element, causing the cognizance of, or the inability to engage in, the undesirable behavior. Other embodiments are described and shown.

### CROSS-REFERENCE TO RELATED APPLICATIONS

This application claims the benefit of provisional patent application Ser. No. 62/800,552, filed 2019 Feb. 12 by the present inventor.

### BACKGROUND

### Prior Art

The following is a tabulation of some prior art that presently appears relevant:

U.S. Patents

| Patent Number | Kind Code | Issue Date | Patentee |
|---|---|---|---|
| 7,899,545 | B2 | 2011 Mar. 1 | John |
| 9,241,665 | B2 | 2016 Jan. 26 | deCharms |

U.S. Patent Application Publications

| Publication Nr. | Kind Code | Publ. Date | Applicant |
|---|---|---|---|
| 20190029584 | A1 | 2019 Jan. 31 | Carr-Jordan |
| 20120293323 | A1 | 2012 Nov. 22 | Kaib |

Foreign Patent Documents

| Foreign | | | | App or |
|---|---|---|---|---|
| Doc. Nr. | Cntry Code | Kind Code | Pub Dt. | Patentee |
| 108784717 | CN | A | 2018 Nov. 13 | Wuxi Peoples |
| | | | | Hospital |

### Nonpatent Literature Documents

Sung, Michael, Massachusetts Institute of Technology. Dept. of Electrical Engineering and Computer Science., “Non-invasive wearable sensing systems for continuous health monitoring and long-term behavior modeling” (October 2005)

### Field

The present invention relates to systems, methods, and articles for providing psychiatric disorder treatment, and more particularly to devices that allow a user to have their mood measured and treatment administered based upon user needs and on information obtained from system input.

### Background

Stories, myths, and religious beliefs reveal the powerful role that anger has played in human affairs since the beginning of recorded history. Explosive outbursts of anger and violence that are inappropriate for the situation at hand cause myriad negative consequences to mental, emotional, physical, and societal well being.

Intermittent explosive disorder (IED) is a particularly acute version of emotional outbursts. The term “intermittent explosive disorder” was first introduced as a psychiatric diagnosis related to impulse dyscontrol in the Diagnostic and Statistical Manual of Mental Disorders (DSM-III 1980).

A national sample in the United States estimated that 16 million Americans may fit the criteria for IED. Lifetime and 12-month prevalence estimates of DSM-IV IED are 7.3% and 5.4% respectively, with a mean of 43-lifetime attacks resulting in $1359 in property damage. Intermittent explosive disorder-related injuries occur 180 times per 100-lifetime cases. Mean age at onset is 14 years., source: Ronald C. Kessler, Ph.D.; Emil F. Coccaro, MD; Maurizio Fava, MD; et al, Arch Gen Psychiatry, “The Prevalence and Correlates of DSM-IV Intermittent Explosive Disorder in the National Comorbidity Survey Replication” (June 2006).

The distinctive feature of impulse control disorders is the failure to resist an impulse, drive, or temptation to perform an act that is harmful to self or others. In diagnosed IED cases, impulsive aggression is not premeditated. Behavior is further defined by a disproportionate reaction to any provocation, real or perceived.

For much of human history, behavioral disorders were either largely ignored or severely punished. This changed when psychotherapy was probably first developed in the Middle East during the 9th century by the Persian physician and psychological thinker, Rhazes. In the West, however, serious mental disorders were still generally treated as demonic or medical conditions requiring punishment and confinement until the advent of moral treatment approaches in the 18th century. Modern psychotherapy does provide an alternative to other older methods, however, fewer than 20% of IED sufferers have been treated in this manner for their impulsive rage attacks.

Treatment is sometimes attempted through psychotropic medication regimens, though the current pharmaceutical options have shown limited success. Anxiolytics help alleviate tension and sometimes help reduce explosive outbursts by increasing the provocative stimulus tolerance threshold and are especially indicated in patients with the commonly comorbid obsessive-compulsive or other anxiety disorders. However; certain anxiolytics are known to increase anger and irritability in some individuals, especially benzodiazepines.

Other anger management techniques include relaxation techniques, monitored breathing exercises, cognitive restructuring, problem-solving, and improving communication strategies and interpersonal skills. However; these treatment types entail significant time and consistency to even produce highly mixed results depending on the diagnosed individual.

Thereafter; several types of user-controlled systems were designed to provide medical responses in such a way as to create a more personalized treatment. John discloses a system for medical monitoring and treatment which can be operated with semi-automatic adjustments; however; the requirement for a patient to manually respond to an alarm makes the system impractical to the sudden, and unconscious nature of an anger outburst.

deCharms shows a system for the measurement of mental states that has an interface that could be used to display therapeutic instructions; however; the relative complexity of the system is cost-prohibitive for the large number needed to be manufactured. Also, with the need of the user to be in direct possession of the device, it limits the usefulness to the sufferer of chronic emotional outbursts who most likely has other debilitating diagnoses that add to disorganization and forgetfulness.

Although systems with a manual response user interface are easy to use and generally less expensive when compared to license professional sanctioned treatments, provide a high degree of personalization, and they provide the flexibility conventional treatments don't, nevertheless they simply do not possess the autonomous, acute counter needed by the many extreme anger outbursts experienced regularly by a large percentage of the human population.

Several types of unique systems have been proposed—for example, in CN patent 108784717 to Wuxi People's Hospital (2018) plus U.S. patents 20,190,029,584 to Can-Jordan (2019) and 20,120,293,323 (2012) to Kaib. Although composed of cutting edge technology, with highly unique approaches, such systems contain many extraneous elements for myriad applications. Thus, the result is a higher manufacturing price, less overall reliability, and accordingly a more difficult degree of access and usage.

My own patent 62/800,552 (2019) shows a system that is constructed to look similar to modern ornamentation, to encourage consistent utilization. Furthermore, the anger outburst systems and methods heretofore known suffer from several disadvantages:

(a) In the case histories of criminal offenders, behavioral therapy has shown to create a higher likelihood of increased recidivism as offenders often find it easier to manipulate others and therefore commit a re-offending crime. Also, if the offender has yet to experience a sufficiently severe outburst, misdiagnosis often occurs. In this circumstance, inappropriate anger management measures are sometimes enforced by authorities, as there is no substantial evidence of a significant link between anger and non-violent offenses.

(b) In a similar manner to the criminal offender who is compelled by external legal pressures, the voluntary drug treatment user is sometimes prescribed counter-productive medicine, or unintentionally over medicate. The activating and mobilizing effects of a healthy amount of emotional anger could be neutralized by sedating chemicals at a dangerous moment needing a responsive fight or flight mechanism. In these instances, an appropriate and valid trigger of anger could be useful, or even life-saving.

(c) The medical treatment in present use is almost entirely non-autonomous. If greater concentration and awareness were present during an extreme anger outburst, this would be less relevant. Often those experiencing an IED outburst are substantially unconscious, as made evident by the recurrent feelings of shame and remorse reported by the suffering individual. Therefore, a solution to these outbursts dictates a largely automatic approach with minimal user intervention to have an acceptable success rate.

(d) The services of a competent anger management psychiatrist are both time and cost-prohibitive in the majority of individuals. Those diagnosed with behavioral disorders, in particular, tend to struggle with these disadvantages due to the high prevalence of co-diagnoses such as generalized anxiety disorder, manic depressive disorder, and attention deficit hyperactivity disorder.

(e) The already uncertain efficacy of current psychological and psychiatric models is further complicated by many patients' lack of compliance with therapy and medication. More than most individuals, those with mental illness or other mood disorders often feel stigmatized as a result of publicly apparent therapeutic activities participated in. This will create further hesitancy in fully embracing the current treatment methods established.

(f) The side effects as a result of taking prescribed medication are often more troublesome to the user than any alleviation of symptoms justify. Depending on the user's healthcare and financial status, medication procurement is often overbearing to the potential user, depriving any lasting improvements such drugs could potentially provide.

### SUMMARY

In accordance with one embodiment, a wearable and autonomous emotional outburst management device system comprises a wearing belt, a detecting sensor housed within the wearing belt, and a preventing member contiguous with the sensor configuration.

### BRIEF DESCRIPTION OF THE DRAWINGS—FIGURES

In the drawings, closely related figures have the same number but different alphabetic suffixes.

FIG. 1 shows a block diagram depiction of a wearable and autonomous emotional outburst management device system in accordance with one embodiment;

FIG. 2 shows a left perspective view of a wearable device that will be attached to a user in accordance with another embodiment;

FIG. 3 shows a right perspective view of a wearable device including several aggregate system components in accordance with another embodiment;

FIGS. 4A to 4O show exploded views demonstrating wearable devices containing various deterring members in accordance with other embodiments;

FIG. 5 shows an exploded view demonstrating the composite members of a power source element in accordance with another embodiment;

FIG. 6 shows a flowchart displaying sequential steps of a system used to manage a user's emotional outbursts in accordance with other embodiments;

FIG. 7 shows a flowchart displaying processes of a system used to manage a user's anger outbursts in accordance with other embodiments;

FIG. 8 shows a system as it functions in terms of input and output in accordance with other embodiments;

FIG. 9 shows a system as it functions in terms of input methods in accordance with other embodiments;

FIG. 10 shows a system as it functions in terms of output methods in accordance with other embodiments; and

FIG. 11 shows a birdseye view demonstrating a wearable device worn by a user in accordance with other embodiments.

Drawings-Reference Numerals

| | | |
|---|---|---|
| 12 wearable housing | 16 connection strap | 20 microcontroller |
| 24 frame | 26 adjustment hole | 28 power source |
| | | element |
| 30 charging jack port | 32 power converter | 34 status monitor |
| 36 male connector | 38 male to male wire | 40 selection element |
| 44 prong | 46 adjustment belt | 101 sensor array |
| 1300 deterring member | | |

### DETAILED DESCRIPTION

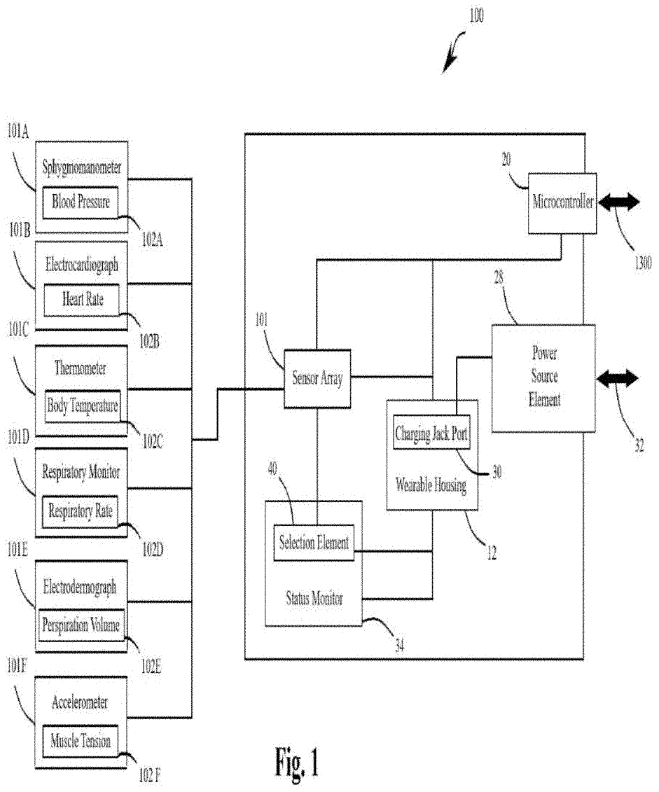

FIG. 1 is a block diagram depiction of a wearable and autonomous emotional outburst management device system 100. It uses a sensor array 101 from direct user bodily contact in an embodiment such as a sphygmomanometer 101A, an electrocardiograph 101B, a thermometer 101C, a respiratory monitor 101D, an electrodermal graph 101E, or an accelerometer 101F.

A sensor array 101 receives a correlated analyte 102 in an embodiment such as blood pressure 102A, heart rate 102B, body temperature 102C, respiratory rate 102D, perspiration volume 102E, or in an embodiment such as muscle tension 102F.

Input reading settings are pre-determined by a selection element 40 relayed to a microcontroller 20 and confirmed by a status monitor 34. A microcontroller 20 analyzes biometric signals in order to send an appropriate response to a deterring member 1300 thereby activating a deterring member 1300.

Power for a wearable and autonomous emotional outburst management device system 100 is received by a power converter 32 that regulates electricity attached to a power source element 28 that is mated into a charging jack port 30 integrated into a wearable housing 12 which allocates power to a system.

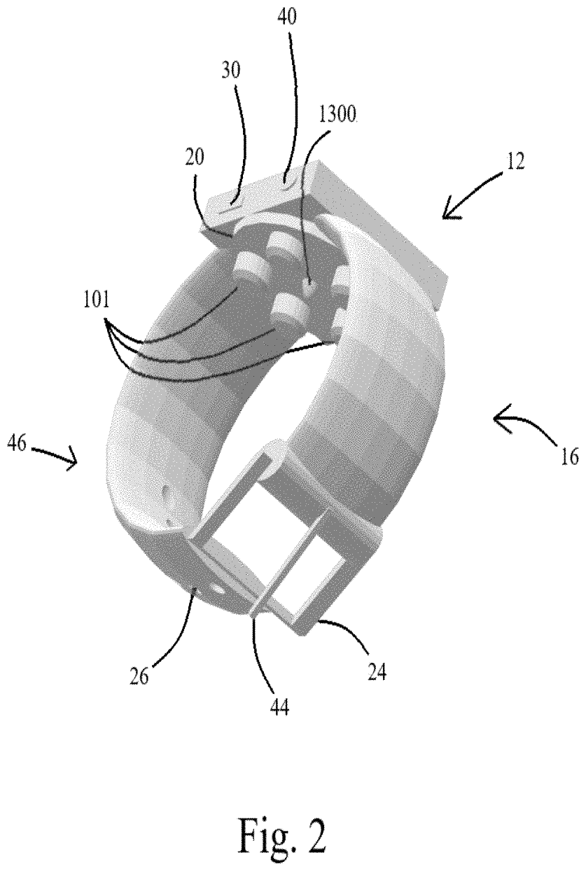

FIG. 2 is a left perspective view of an embodiment of a wearable device that will be attached to a user via an adjustment belt 46 sheathed by a frame 24. A frame 24 is an end component of a connection strap 16 that houses a prong 44. A prong 44 is thrust into an adjustment hole 26 as a feature of an adjustment belt. At present, I contemplate the use of silicone for an adjustment belt and connection strap components, but other materials are suitable.

A sensor array 101 connects to a microcontroller 20 activated by internal control systems which transmit information to a deterring member 1300 that activates a deterring member 1300. A visual display is located on a status monitor 34 arranged vertically atop a selection element 40. A selection element is horizontally aligned with a charging jack port 30. A microcontroller 20, status monitor 34, selection element 40, and charging jack port 30 are all fused to a wearable housing 12.

At present, I contemplate the use of modern-day cellular telephone building materials for construction of a wearable housing 12. These would include aluminum oxide, silicon dioxide, indium tin oxide, aluminum alloys, lithium cobalt oxide, carbon graphite, gold, copper, silver, platinum, tungsten, neodymium-iron-boron alloys, dysprosium and praseodymium, and polycarbonate plastic, but other materials are suitable.

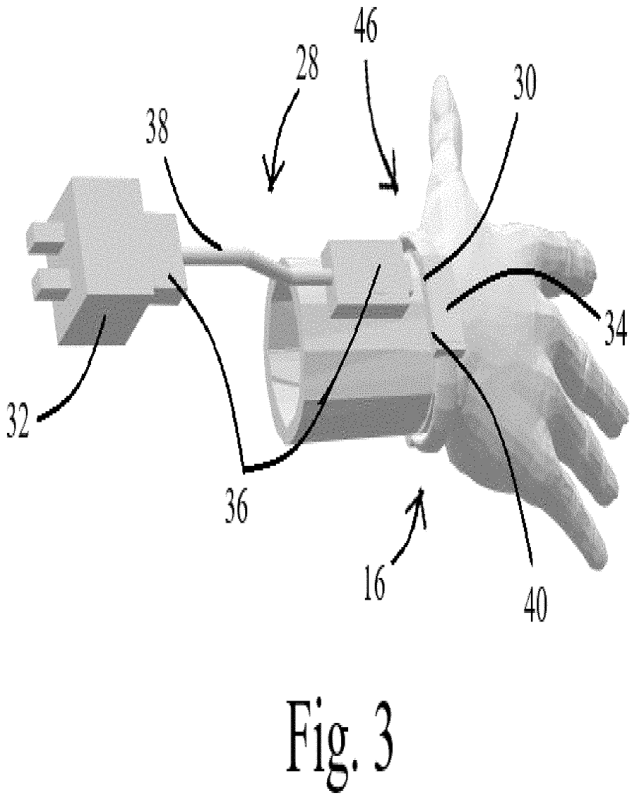

FIG. 3 is a right perspective view of an embodiment of a wearable device including several aggregate system components. A power converter 32 accepts a male connector 36. A male connector 36 is fused to a male to male wire 38. A male to male wire 38 terminates at a male connector 36.

A power source element 28 connects to a charging jack port 30. A charging jack port 30 is held static simultaneously by an adjustment belt 46 and a connection strap 16. Successful power transmission is displayed by a status monitor 34 and controlled by a user via a selection element 40.

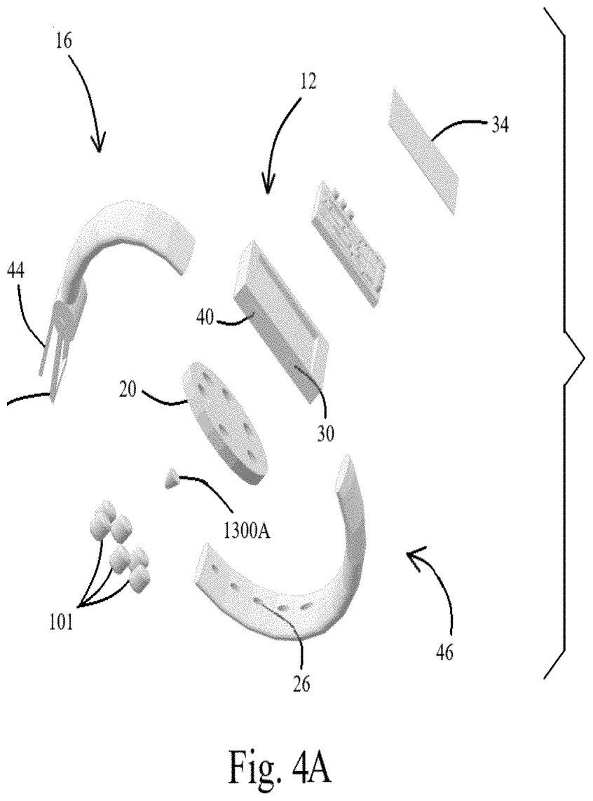

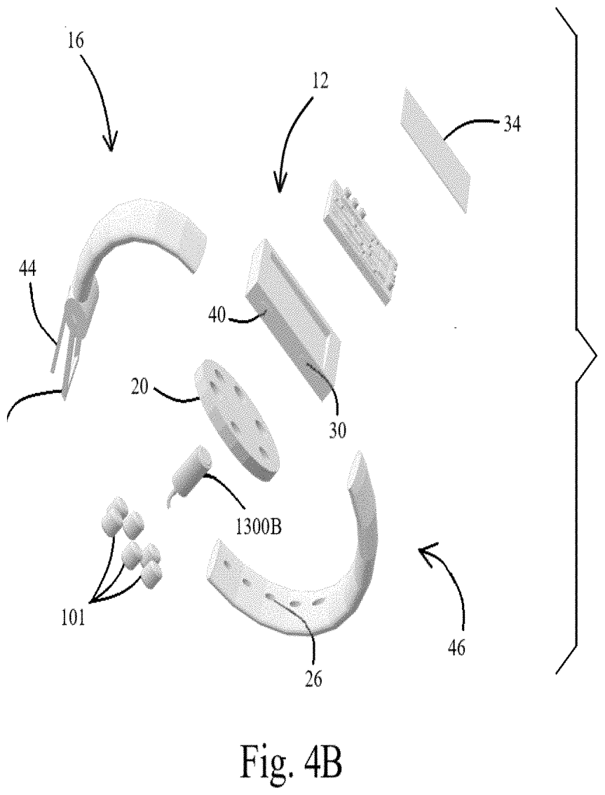

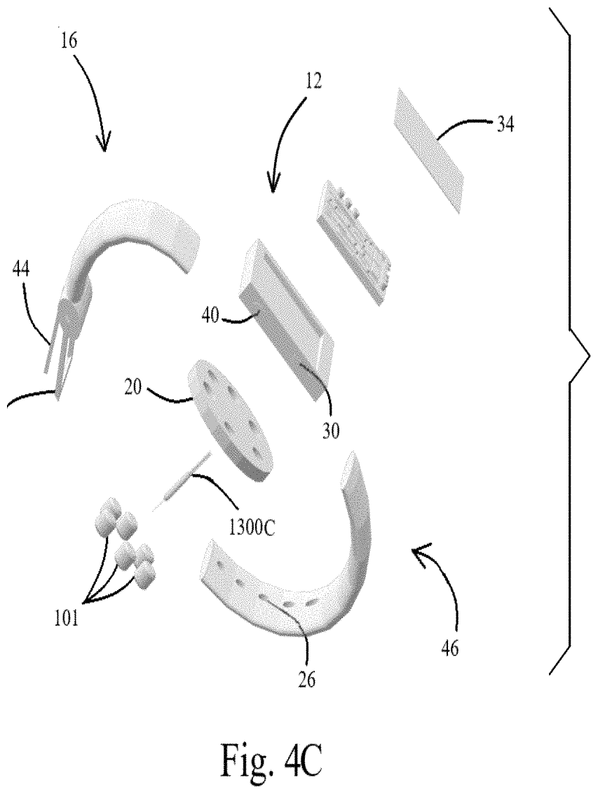

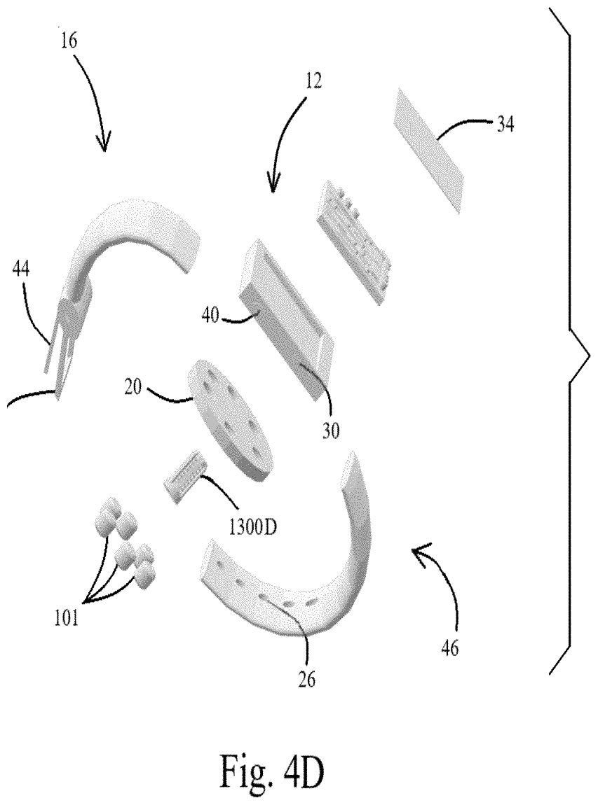

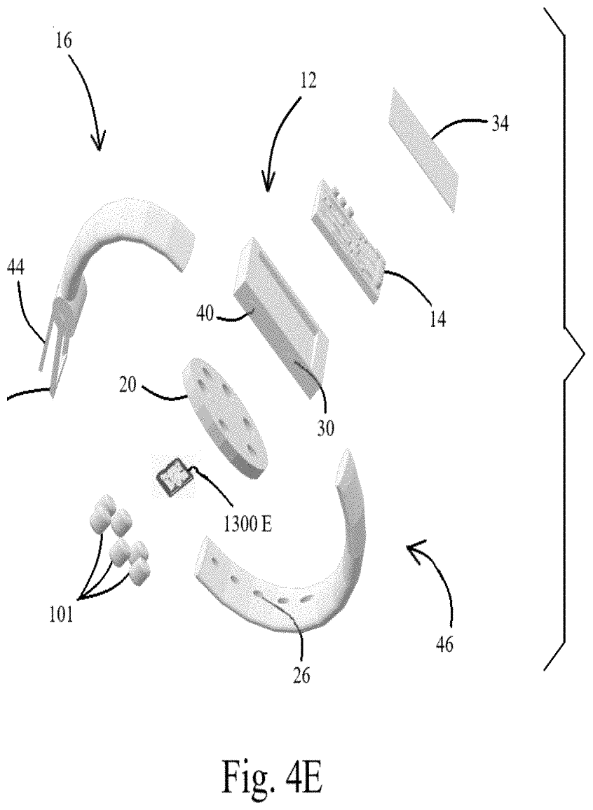

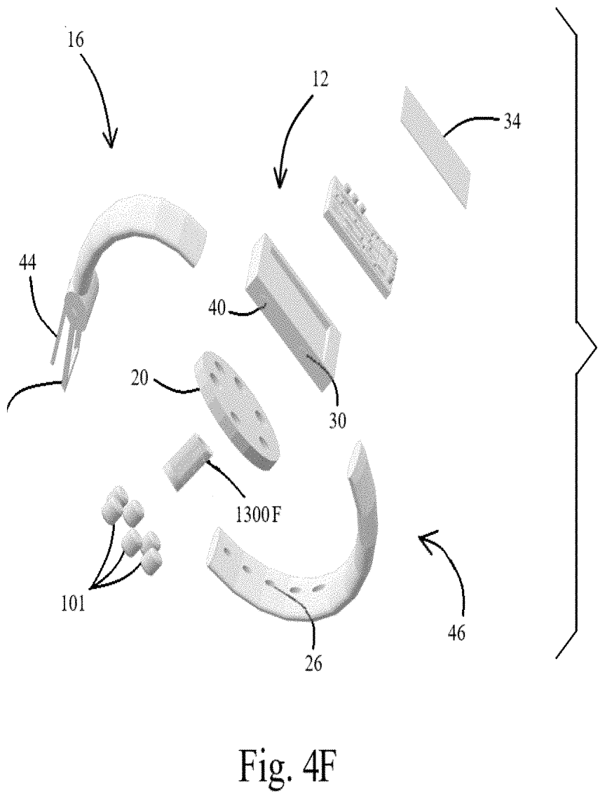

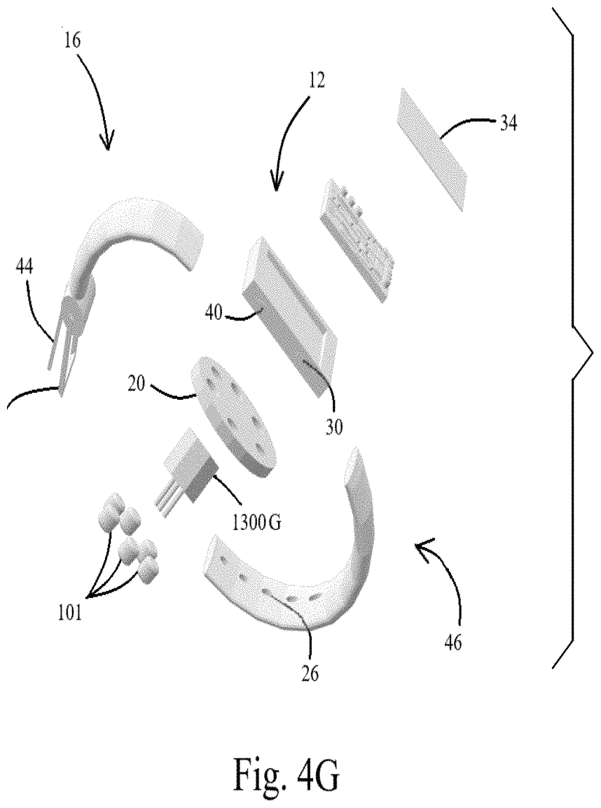

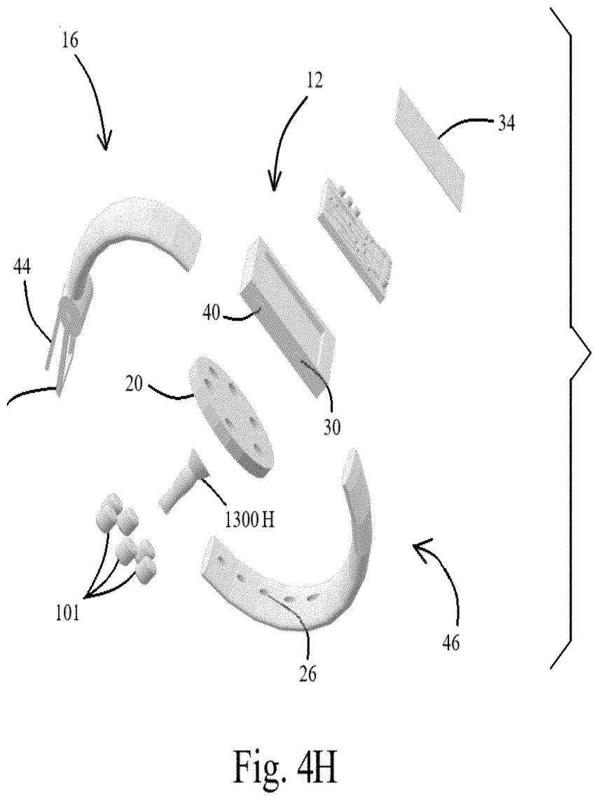

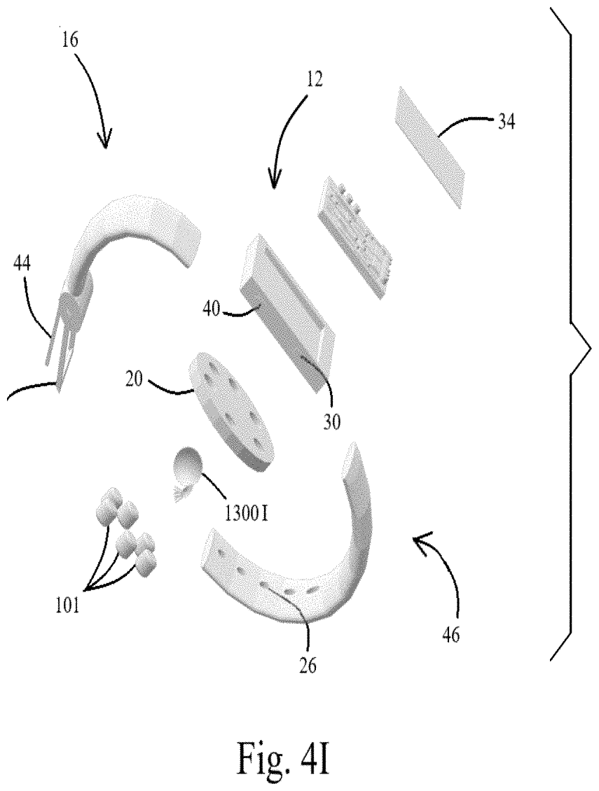

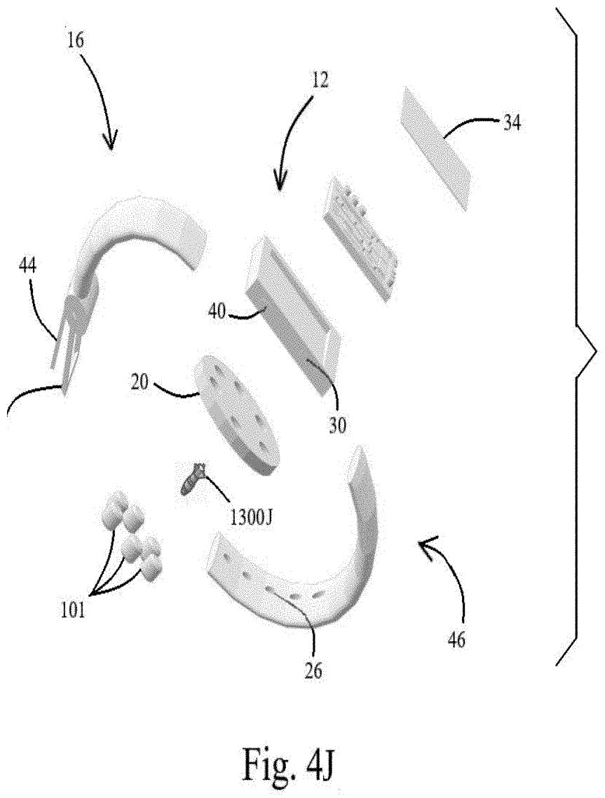

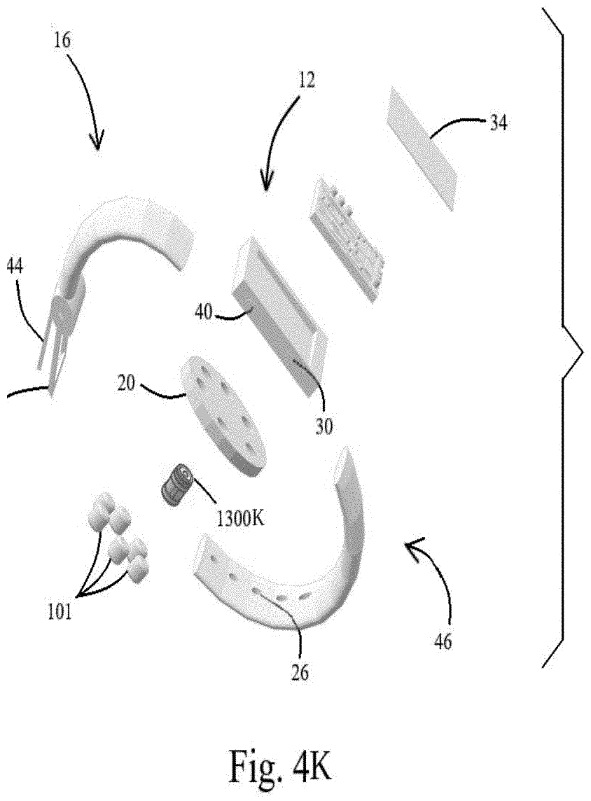

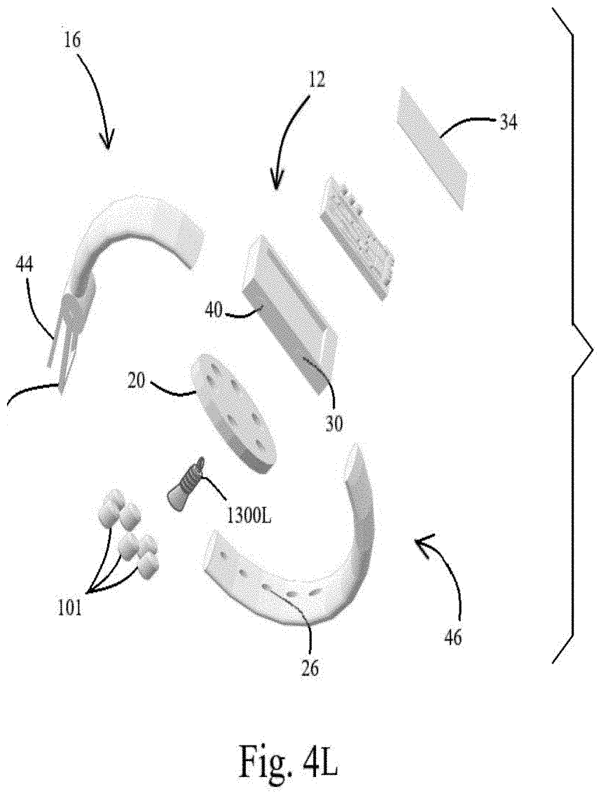

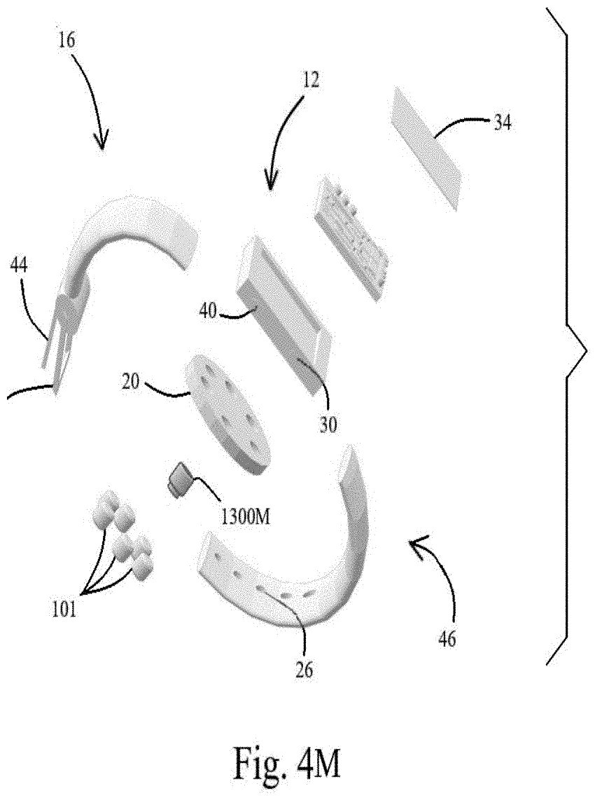

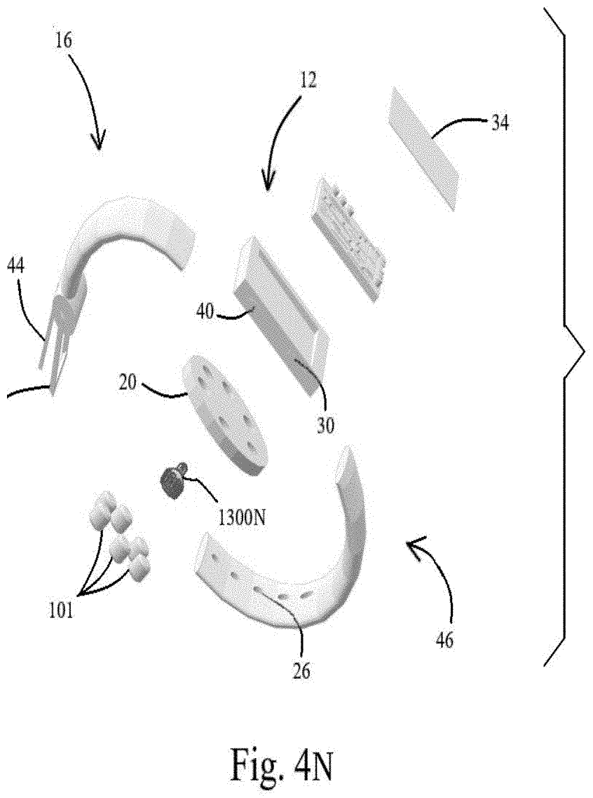

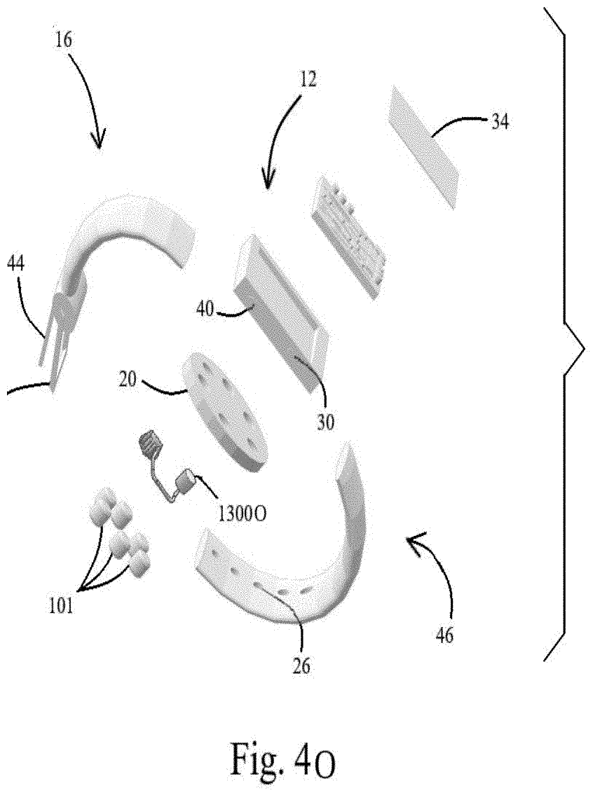

FIG. 4A is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as an electroshock contact 1300A as a method for delivering conducted electricity up to 50,000 volts (est), 26 watts, 3.6 milliamperes (Irms), and 0.50 Joules per pulse energy.

FIG. 4B is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a lachrymatory spray canister 1300B as a method for delivering oleoresin capsicum (OC) spray up to 1.33% concentration major capsaicinoids.

FIG. 4C is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a paralytic needle 1300C as a method for delivering singular or combination of liquified anxiolytic and antipsychotic medication as prescribed.

FIG. 4D is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a tacky propellant hose 1300D as a method for delivering formulation SF-283 (sticky foam) pressurized up to 344.7379 kPa.

FIG. 4E is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as an emetic patch 1300E as a method for delivering extraction of the plants Cephaelis acuminata and Cephaelis ipecacuanha (ipecac) up to 2.5% concentration.

FIG. 4F is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a directed radiation laser 1300F as a method for delivering Diode-pumped solid-state laser with green light output configured to 532 nanometers (nm).

FIG. 4G is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as an electromagnetic radiation gun 1300G as a method for delivering microwave frequency range up to 300 gigahertz (GHz), having wavelengths up to 1 meter.

FIG. 4H is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a malodorant fogger 1300H as a method for delivering an organic and non-toxic blend of baking powder, yeast, and other ingredients (commonly referred to as “skunk”).

FIG. 4I is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a sound pressure amplifier 1300I as a method for delivering sound pressure up to 100 decibels (dB).

FIG. 4J is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a blunt shrapnel explosive 1300J as a method for delivering blunt pellets with explosive content up to 10 grams.

FIG. 4K is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a net entrapment projector 1300K as a method for delivering polyester net up to 5 meters by 5 meters, with a double weave fabric strength up to 1050 denier.

FIG. 4L is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a light-emitting diode (LED) incapacitator 1300L as a method for delivering light with up to 100 luminance (lum) with effective distance up to 3 meters (m).

FIG. 4M is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a vesicant dripper 1300M as a method for delivering phosgene oxime (CX) dose-drips of up to 3 mg.min/m3.

FIG. 4N is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as a sensory-disorienting munition 1300N as a method for delivering munition releasing up to 170 decibels (dB) of sound pressure and 6 million candela of light up to 1.5 meters (m) away.

FIG. 4O is an exploded view demonstrating capabilities of an embodiment of a wearable device composed of a wearable housing 12 with an adjoining charging jack port 30, a protuberance of a selection element 40, fused to a connection strap 16, and an adjustment belt 46 constructed in accordance with an embodiment.

A sensor array 101 within an array bores through a microcontroller 20, creating a connection to a microcontroller 20 which is contained within a wearable housing 12. A sensor array 101 relays acquired physiological measurements to a status monitor 34. An embodiment is secured by a frame 24 colligated with a prong 44 thrust into an adjustment hole 26. An embodiment containing a stimulus actuator is depicted as an anesthetic gas mask 1300O as a method for delivering up to 70% vapors of nitrous oxide, desflurane, isoflurane and sevoflurane combined and at least 30% oxygen delivery.

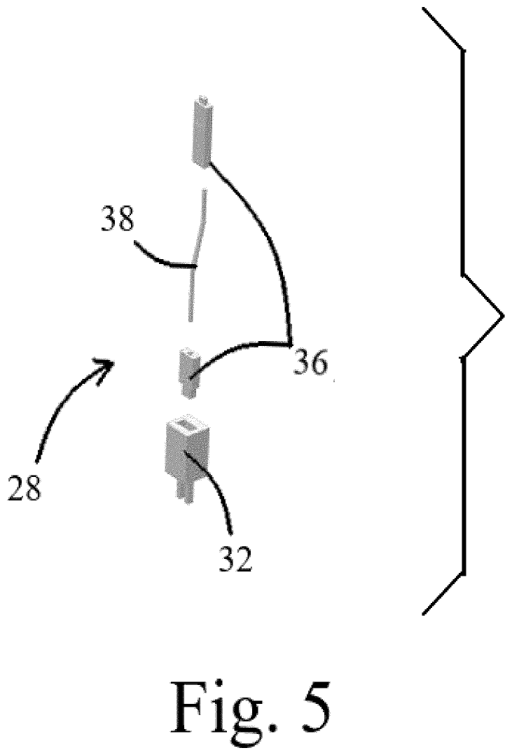

FIG. 5 is an exploded view demonstrating an embodiment with an electrical powering attachment connected to an embodiment composed of a power source element 28 containing a power converter 32, and a repeating male connector 36 at opposing ends connected by a male to male wire 38.

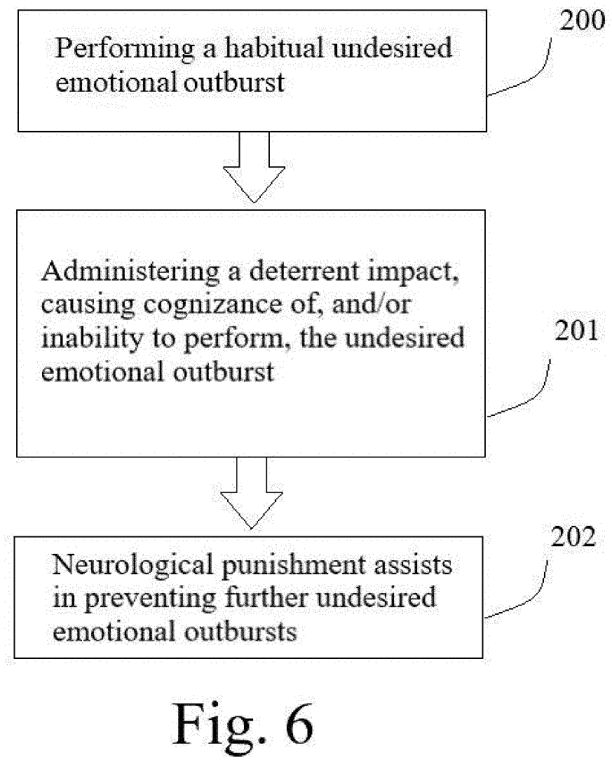

FIG. 6 is a flowchart showing the sequential steps of a user performing a habitual undesired emotional outburst 200, an administering a deterrent impact, causing cognizance of, and/or inability to perform, the undesired emotional outburst 201, and the resulting neurological punishment assists in preventing further undesired emotional outbursts 202 using an embodiment of a system used to manage a user's anger outbursts.

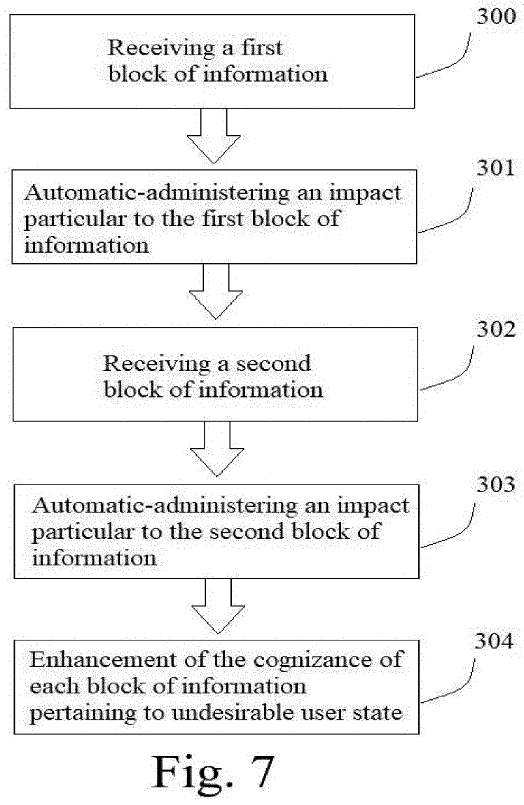

FIG. 7 is a flowchart showing steps for an embodiment of a system used to manage a user's anger outbursts, wherein, a system is receiving a first block of information 300, actuating the automatic-administering of an impact particular to the first block of information 301, whilst simultaneously receiving a second block of information 302, actuating the automatic-administering of an impact particular to the second block of information 303, resulting in an enhancement of the cognizance of each block of information pertaining to undesirable user state 304 that is an aggressive and disproportionate reaction to the situation at hand.

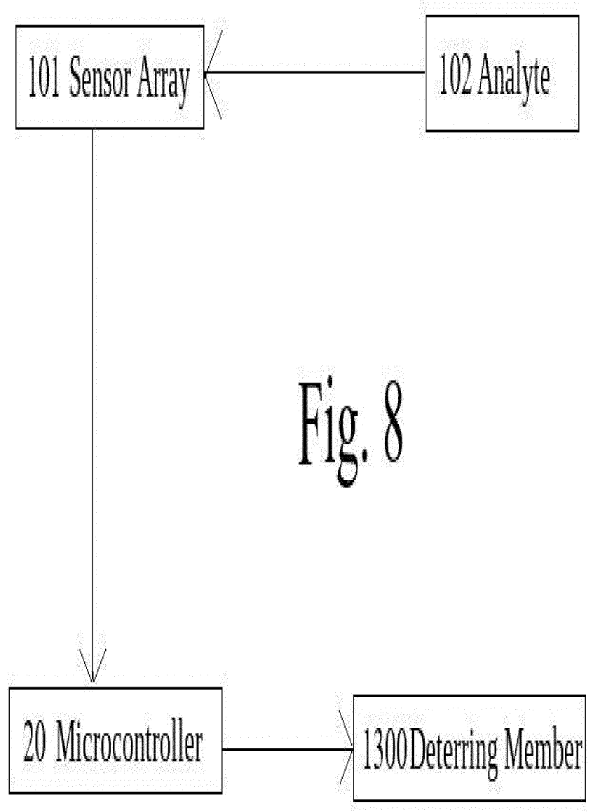

FIG. 8 is a system as it functions in terms of input from an analyte 102 to a sensor array 101, and output from a microcontroller 20 to a deterring member 1300.

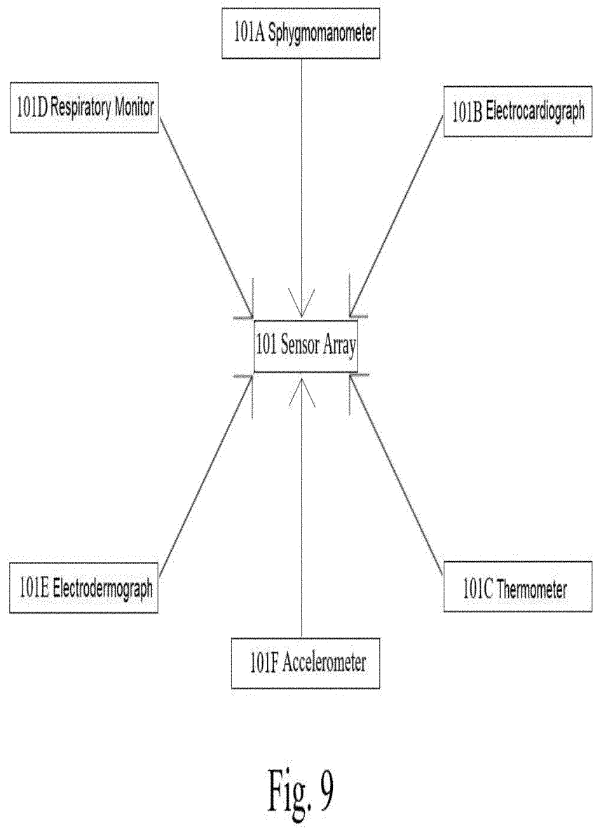

FIG. 9 is a system as it functions in terms of a sensor array 101, comprising at least one sphygmomanometer 101A, electrocardiograph 101B, thermometer 101C, respiratory monitor 101D, electrodermal graph 101E, or accelerometer 101F.

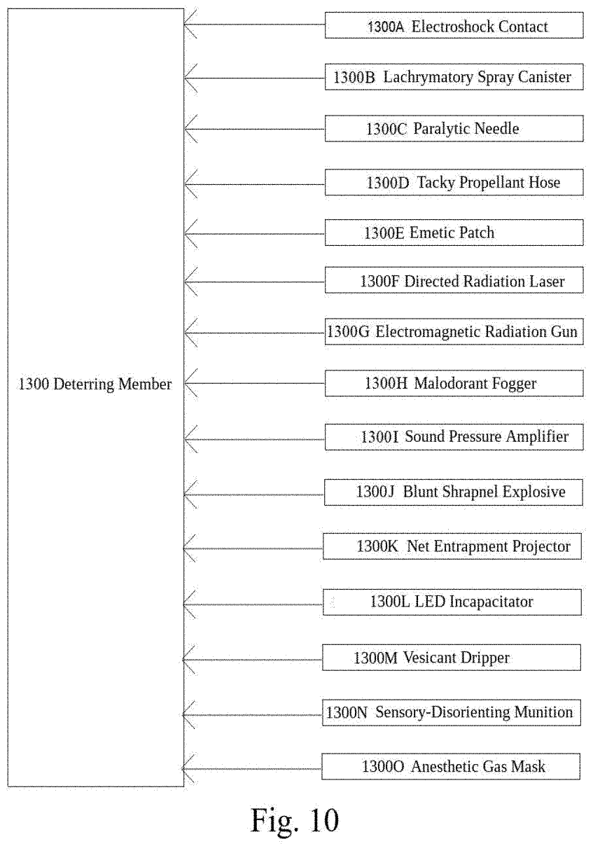

FIG. 10 is a system as it functions in terms of a deterring member 1300, comprising at least one electroshock contact 1300A, lachrymatory spray canister 1300B, paralytic needle 1300C, tacky propellant hose 1300D, emetic patch 1300E, directed radiation laser 1300F, electromagnetic radiation gun 1300G, malodorant fogger 1300H, sound pressure amplifier 1300I, blunt shrapnel explosive 1300J, net entrapment projector 1300K, light-emitting diode (LED) incapacitator 1300L, vesicant dripper 1300M, sensory disorienting munition 1300N, or anesthetic gas mask 1300O.

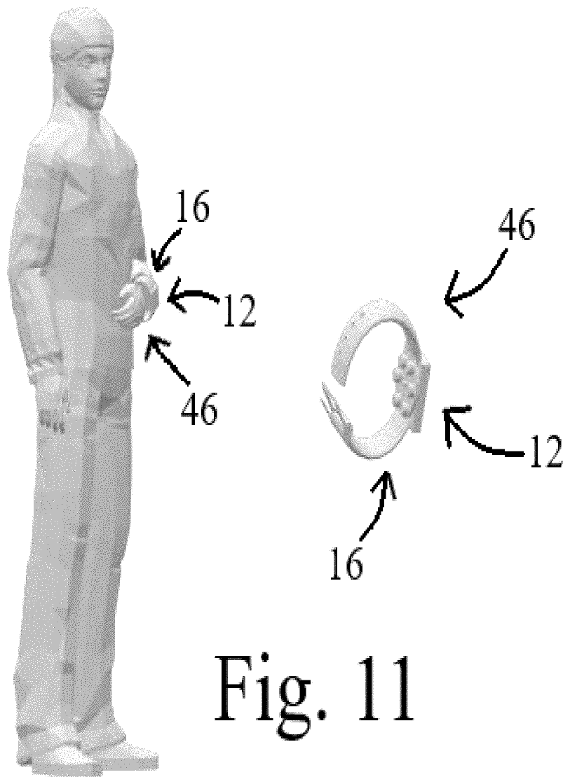

FIG. 11 is a birdseye view demonstrating an embodiment of a wearable device worn by a user composed of a wearable housing 12, connection strap 16, and adjustment belt 46.

Operation

In operation, one uses an embodiment of my wearable and autonomous emotional outburst management device system by attaching the wearable housing 12 (FIG. 2) in a normal manner by attaching the connection strap 16 to the adjustment belt 46 (FIG. 2). Prior to this the user adequately charges the wearable housing 12 using the connected power source element 28 (FIG. 3). An embodiment is worn by a user to assist in anger outburst subjugation.

As displayed in FIG. 8, an embodiment receives one or more analyte 102 of the human user through bodily contacting one or more sensor array 101. These readings are sent to the microcontroller 20 contained in the wearable housing 12. The microcontroller 20 processes the information then analyzes the biometric signals. With the biometric signals analyzed, the microcontroller 20 determines an appropriate response or lack of response.

If a response is warranted, the deterring member 1300 attached to the microcontroller 20 will react appropriately. When these predetermined conditions manually selected from selection element 40 occur, the user will be affected by the deterring member 1300; ranging from sudden awareness to total incapacitation. The severity of the deterrent output will differ depending on the needs of the user.

FIG. 2 displays a method in which the user can control the operation of the embodiment by manually administering stimuli via the selection element 40. The system automatically produces interruption stimuli in response to one or more measurements of an analyte 102 acquired by a sensor array 101 (FIG. 8) that indicates an imminent anger outburst.

The user can, when desired, activate the status monitor 34 (FIG. 3) by pressing the selection element 40 to activate the microcontroller 20 (FIG. 4A). When the status monitor 34 is activated, the selection element 40 can be engaged by the user to set when predetermined conditions occur, as well as, display and adjust the following metrics:

(1) As shown in FIG. 1, one of A sensor array 101 receiving biofeedback consisting of a sphygmomanometer 101A, an electrocardiograph 101B, a thermometer 101C, a respiratory monitor 101D, an electrodermal graph 101E, or an accelerometer 101F.

(2) As shown in FIG. 1, one of an analyte 102 reading consisting of blood pressure 102A, heart rate 102B, body temperature 102C, respiratory rate 102D, perspiration volume 102E, or muscle tension 102F.

(3) As shown in FIG. 10, one of a deterring member 1300 fused to the microcontroller 20 (FIG. 2) provides a deterrent consisting of an electroshock contact 1300A, a lachrymatory spray canister 1300B, a paralytic needle 1300C, a tacky propellant hose 1300D, an emetic patch 1300E, a directed radiation laser 1300F, a electromagnetic radiation gun 1300G, an malodorant fogger 1300H, or an sound pressure amplifier 1300I.

One or more analyte 102 (FIG. 8) digital readings are the measurable physiological changes that occur in the user's body leading up to the cause of physical damage to people or objects around the user due to intermittent explosive disorder diagnosis or other anger-related mental illness.

Settings for the microcontroller 20 and an associated deterring member 1300 are determined via the selection element 40 (FIG. 2) and status monitor 34 (FIG. 3). After baseline user's measurements are taken and stimulus output is determined, the embodiment calls for nominal effort from the user.

Advantages

From the description above, a number of advantages of some embodiments of my behavioral treatments become evident:
(a) They provide behavioral treatments that are more convenient.
(b) They reduce the need for unpredictable medication effects.
(c) They do not entail the significant time and consistency commitment of other treatments.
(d) They operate substantially autonomously, plus they are simpler and more reliable.
(e) They are more accessible and inexpensive to manufacture.
(f) They will be used with a minimal of encumbrance, providing at the same time a persistent and tangible reminder.
(g) They provide potent external punishment often justified by many extreme anger outbursts such as experienced by those meeting the IED criteria.
(h) They are designed specifically to treat the most potentially destructive and common drive in human beings.
(i) They do not rely on inconsistent psychotherapy intervention, instead offering to empower the user with a level of involvement.
(j) They treat the conditioned anger response in a noninvasive and rapid manner so the user will be more motivated to continue treatment.
(k) They allow flexibility in the choice of the deterrent(s), allowing for a broader user base.
(l) They are configured to have detecting implementation customized according to a user, guardian, parole officer, health official, or other third party's unique circumstances.
(m) They allow readily available observation regarding the level of the users' compliance and treatment effectiveness in comparison to current psychological and psychiatric models.
Other advantages of one or more aspects will be apparent from a consideration of the drawings and ensuing description.

Conclusion, Ramifications, and Scope

Thus, the reader will see that at least one embodiment provides a more convenient, reliable, yet efficient solution to anger outbursts than current methods relying on therapy and medication. Additionally, the anger treatments heretofore known demand that the user engages in continual self-monitoring and repeated visits to medical professionals.

However, a user experiencing the physiological and environmental effects of anger needs an automatically triggered system that operates autonomously to avoid extreme harm to self or others. The wearable and autonomous emotional outburst management device system embodiment delivers immediate, dependable, and potentially incapacitating capabilities in a reliable manner. Such a solution will save many material objects, relationships, and lives.

Although the description above contains many specificities, these should not be construed as limitations on the scope, but rather as an exemplification of one of several embodiments thereof. Many other variations are possible. For example, the adjustment belt, connection strap, frame, adjustment hole, or prong can have other shapes, such as circular, oval, trapezoidal, triangular, etc.; the wearable housing can be made of materials that increase or decrease the potency of the deterrence, such as conductive, absorbent, synthetic, natural, etc.; the frame can additionally include semi-permanent glue, magnets, snapping buttons, or be surgically fastened; the sensor array can include additional biological parameters to monitor such as deglutition, desquamation, egestion, eructation, lacrimation, manducation, micturition, pandiculation, protopepsia, sialorrhea, stasis, sternutation, or vomiting; the status monitor can have a predetermined numeric sequence input involved to attach or remove the system, etc.

Thus the scope of the embodiments should be determined by the appended claims and their legal equivalents, rather than by the examples given.

I claim:

1. A wearable and autonomous emotional outburst management device system, comprising:
a. wearing means, including a wearable housing having a predetermined rectangular shape and incorporating one or more locking mechanisms, to connect said wearable and autonomous emotional outburst management device system to a user;
b. detecting means, including one or more sensors housed within said wearable means configured to monitor one or more indicators sensing the physiological parameters of said user, thereby communicating the current anger status of said user; and
c. preventing means, including at least one sense depriving member contiguous with said detecting means configured to release one or more deterring member having predetermined parameters that will cause substantially instant cessation of intermittent explosive disorder (IED) episodes;
whereby said wearable and autonomous emotional outburst management device system prevents said intermittent explosive disorder (IED) episodes from manifesting, using non-fatal physical punishment as a way of bringing about substantial cognizance or subduing completely, said user in near real-time.

2. The system of claim 1 further comprising said first means entails inputting a predetermined numeric sequence registered on a user interface to attach or remove said wearable and autonomous emotional outburst management device system.

3. The system of claim 1 wherein said second means is at least one sphygmomanometer to measure the blood pressure of said user.

4. The system of claim 1 wherein said second means is at least one electrocardiograph to measure the heart rate of said user.

5. The system of claim 1 wherein said second means is at least one thermometer to measure the body temperature of said user.

6. The system of claim 1 wherein said second means is at least one respiratory monitor to measure the respiratory rate of said user.

7. The system of claim 1 wherein said second means is at least one electrodermal graph to measure the perspiration volume of said user.

8. The system of claim 1 wherein said second means is at least one accelerometer to measure the muscle tension of said user.

9. An article, comprising:
a. a wearable housing to attach said article to the epidermis of a user;
b. a microcontroller housed in said wearable housing configured to process input and output operations of said article;
c. A sensor array connected to said microcontroller configured to acquire one or more analytes of said user contacting said article; and
d. one or more deterring members connected to said microcontroller to deliver at least one deterrent directed at said user attached to said article;
whereby said at least one deterring member will automatically and preemptively interrupt said user attached to said article from manifesting belligerent behavior when at least one analyte threshold is detected by said sensor array, triggering an instruction to deliver at least one deterrent by said microcontroller in response

10. The article of claim 9 wherein said wearable housing is secured to said user with at least one prong passed through at least one gap.

11. The article of claim 9 wherein said wearable housing is attached to said user with an adhesive.

12. The article of claim 9 wherein said wearable housing is surgically implanted in said user.

13. The article of claim 9 wherein said wearable housing is connected to said user with at least one magnetic element.

14. The article of claim 9 wherein said wearable housing is fastened to said user with at least one metallic button.

15. The article of claim 9 wherein said wearable housing is bound to said user with handcuffs composed of plastic materials or metallic materials.

16. The article of claim 9 wherein said deterring member administers one or more elements including conducted electricity, kinetic blunt force trauma, restraint cords, chemical irritants, tranquilizing drugs, incapacitant materials, emetic compounds, optical blinding elements, electromagnetic radiation, malodorant molecules, or amplified sound waves.

17. A method, comprising:
a. providing first means, for wearing a behavioral treatment device by a user;
b. providing second means, connected to said first means, for acquiring one or more body signals of said user; and
c. providing third means, connected to said first means, for administering one or more adjustable stimuli when predetermined parameters of said body signals of said user are detected;
whereby said first means, second means, and third means respond together dynamically to provide an acute and automatic remedy against said user undesirably altering substantial surroundings.

18. The method of claim 17 wherein said first means is at least one restraining system that can be removed by at least one other user or at least one other object.

19. The method of claim 17 wherein said second means is at least one biosensor.

20. The method of claim 17 wherein said third means is at least one non-lethal weapon including a blunt shrapnel explosive, net entrapment projector, light-emitting diode (LED) incapacitator, vesicant dripper, sensory-disorienting munition, anesthetic gas mask, electroshock contact, lachrymatory spray canister, paralytic needle, tacky propellant hose, emetic patch, directed radiation laser, electromagnetic radiation gun, malodorant fogger, or sound pressure amplifier.
